(19) United States
(12) Patent Application Publication
(10) Pub. No.: US 2005/0119644 A1
(43) Pub. Date: Jun. 2, 2005

(54) Articulating catheter tip with wedge-cuts

(76) Inventor: Richard J. Koerner, San Diego, CA (US)

Correspondence Address: NYDEGGER & ASSOCIATES, 348 OLIVE STREET, SAN DIEGO, CA, 92103, US

(21) Appl. No.: 10/725,148

(22) Filed: Dec. 1, 2003

(51) Int. Cl. A61B 18/18

(52) U.S. Cl. 606/20; 604/95.04

(57) ABSTRACT

An articulating catheter for cryoablating target tissue having a curved surface includes an elongated, thermally conductive tube that has an outer surface and is formed with a plurality of transverse notches. The notches allow the tube to be reconfigurable between a first configuration wherein said tube is substantially cylindrical and a second configuration in which at least a portion of the outer surface of the tube is shaped to substantially conform with the surface of the target tissue.

### FIELD OF THE INVENTION

The present invention pertains generally to surgical instruments. More particularly, the present invention pertains to medical catheters designed to cool contacted body tissue to extremely low (i.e. cryogenic) temperatures. The present invention is particularly, but not exclusively, useful as a cryocatheter having a segment that can be reshaped in situ to contact and cool internal target tissue having a complex surface geometry in a one-step process.

### BACKGROUND OF THE INVENTION

There are many applications in which it is desirable to contact and cool tissue having a complex (e.g. non-flat) surface geometry. One such application is the ablation of a circumferential band of tissue surrounding the ostium of a pulmonary vein where the pulmonary vein connects with the left atrium. This band of tissue can be ablated in a procedure to treat a somewhat common heart ailment known as atrial fibrillation.

Research has shown that atrial fibrillation is due to abnormal electrical signals that pass through (or originate at) the tissue surrounding the ostia of the pulmonary veins where the pulmonary veins connect with the left atrium. Once the circumferential band of tissue surrounding the affected ostium has been ablated, the destroyed tissue is no longer able to initiate or conduct any type of electrical signal. Accordingly, ablation can be used to prevent abnormal electrical signals from the pulmonary veins from reaching the heart.

One technique that has been used to cryoablate the circumferential band of tissue has involved sequentially ablating tissue at a plurality of relatively small locations around the periphery of the ostium. To perform this procedure, the cold cryotip of the cryoablation catheter must be repeatedly moved (i.e. reoriented) to sequentially contact portions within a band of tissue. In theory, these ablations can combine to establish an effective circumferential ablation band. However, in practice, this complex process often results in a non-uniform or discontinuous circumferential lesion that does not adequately block all of the abnormal electrical signals from entering the heart. Moreover, this procedure is time consuming because it requires extensive manipulation of the cryotip around the ostium. The result is a somewhat lengthy procedure that increases patient discomfort and increases the probability that complications may result from the procedure.

The present invention contemplates the cryoablation of a circumferential band of tissue in a single-step (i.e. the entire band of tissue is ablated simultaneously). This requires contacting the circumferential band of tissue with a contacting element having a relatively large-diameter, somewhat cylindrical shaped contact surface. The problem, however, has been the non-invasive delivery of a contacting element having this relatively large, bulky shape to the treatment site. In particular, the human vasculature is curved, branched and contains vessels having relatively small inner diameters. As a consequence, it is necessary to design a catheter having a relatively low profile to allow the distal end of the catheter to navigate through the complex vasculature. With this in mind, it would be desirable for a catheter to have a relatively low profile for transit through the vasculature and a relatively large contact surface to allow for a one-step cryoablation. To solve this dilemma, the present invention contemplates a contacting element that can be reshaped in-situ from a relatively low profile shape to a shape suitable for contacting a circumferential band of tissue.

In light of the above, it is an object of the present invention to provide a system and method for performing a non-invasive, single-step cryoablation of a circumferential shaped band of tissue in the vasculature of a patient. Another object of the present invention is to provide a system and method for treating atrial fibrillation by cryoablating the peripheral tissue surrounding the ostium of a pulmonary vein where the pulmonary vein connects to the left atrium. Still another object of the present invention is to provide a system and method for cryoablating tissue in the vasculature of a patient in a relatively quick, efficient and reliable manner.

### SUMMARY OF THE INVENTION

The present invention is directed to an articulating catheter for cryoablating target tissue at a treatment site. In particular, the articulating catheter can be used to cryoablate target tissue having a curved (i.e. non-flat) surface. For the present invention, the articulating catheter includes an elongated, thermally conductive tube that has an outer surface and is formed with a plurality of transverse notches. With this cooperation of structure, the tube is reconfigurable between a first configuration wherein the tube is substantially cylindrical and a second configuration in which at least a portion of the outer surface of the tube is shaped to substantially conform with the surface of the target tissue.

In greater structural detail, each notch establishes a first edge and an opposed second edge. In the first configuration, each edge is inclined relative to a plane that is substantially perpendicular to a longitudinal axis defined by the cylindrical shaped tube. On the other hand, when the tube is in the second configuration, the first edge of each notch is juxtaposed with the second edge of the notch, and the tube is no longer cylindrical.

In an exemplary embodiment on the articulating catheter, the notches can be configured such that the tube is curved in the second configuration and establishes an inner radius of curvature, ρᵢₙₙₑᵣ, relative to a central axis and an outer radius of curvature, ρₒᵤₜₑᵣ, relative to the central axis. More specifically, the portion of the tube that is distanced from the central axis by the distance ρₒᵤₜₑᵣ, constitutes a continuous, thermally conductive band that can be placed in contact with target tissue having a curved surface and cooled to cryoablate the target tissue.

In a typical arrangement, the tube is configured in the first configuration and attached to a cryo-element having an expansion chamber. A catheter tube is then used to advance the cryo-element and tube to the treatment site whereupon the tube can be reconfigured into the second configuration. For example, a pull-wire attached to the distal end of the tube can be actuated to reconfigure the tube. Once reconfigured, the conforming portion of the tube is placed in contact with the target tissue. Next, a refrigerant can be passed through the catheter tube and expanded in the expansion chamber to cool the cryo-element and tube. The cooling can be continued until the target tissue is effectively cryo-ablated.

### BRIEF DESCRIPTION OF THE DRAWINGS

The novel features of this invention, as well as the invention itself, both as to its structure and its operation, will be best understood from the accompanying drawings, taken in conjunction with the accompanying description, in which similar reference characters refer to similar parts, and in which:

FIG. 1 is a perspective view of a system for cryoablating internal target tissue shown operationally positioned in a patient;

FIG. 2 is perspective view of a distal portion of the cryoablation system shown in FIG. 1;

FIG. 3 is side plan view of a reshapeable contact segment shown in a first configuration in which the contact segment is cylindrically shaped;

FIG. 4 is a front plan view of the reshapeable contact segment shown in FIG. 3;

FIG. 5 is a side plan view of the reshapeable contact segment shown in FIG. 3, shown after reconfiguration into a second configuration in which a portion of the outer surface of the contact segment is shaped to substantially conform with the surface of the target tissue;

FIG. 6 is a cross-sectional view of a distal portion of the cryoablation system shown in FIG. 1, as seen along the line 6-6 in FIG. 2;

FIG. 7 is a perspective view of a distal portion of the cryoablation system shown in FIG. 1, shown in the straight configuration and positioned at a treatment site in the vasculature of a patient; and

FIG. 8 is a perspective view of a distal portion of the cryoablation system shown in FIG. 1, shown in the curved configuration and positioned at a treatment site in the vasculature of a patient.

### DESCRIPTION OF THE PREFERRED EMBODIMENTS

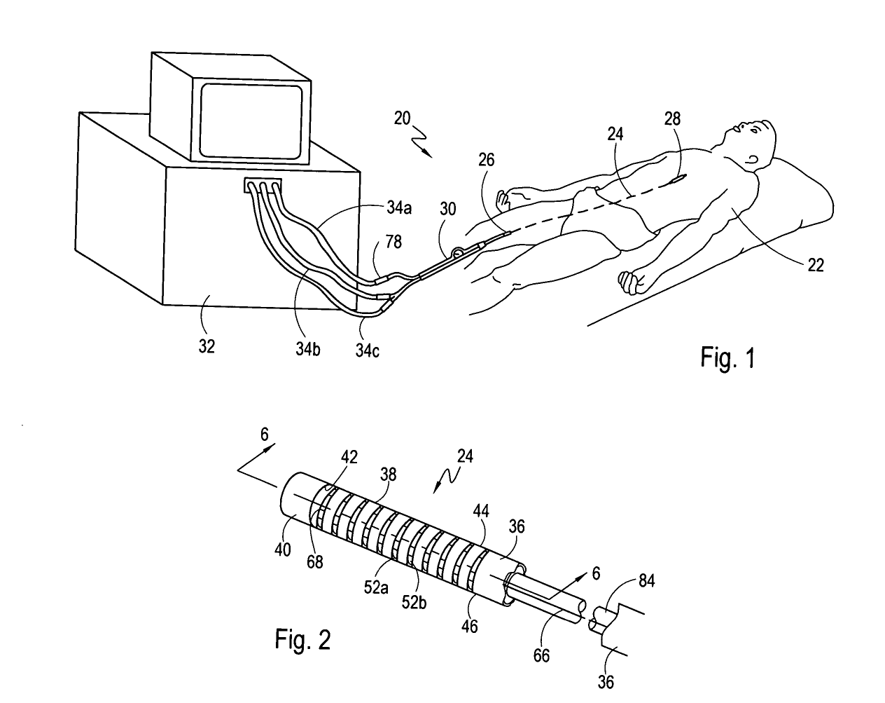

Referring initially to FIG. 1, a system 20 for ablating internal target tissue of a patient 22 is shown. As shown, the system 20 includes a catheter 24 that extends from a proximal end 26 that remains outside the patient's body during the procedure to a distal end 28. From FIG. 1 it can be seen that the distal end 28 of the catheter 24 has been inserted into the patient 22 through an artery such as the femoral artery and advanced through the patient's vasculature until the distal end 28 is positioned in the upper body of the patient 22. FIG. 1 further shows that the proximal end 26 of the catheter 24 is connected to a catheter handle 30, which in turn is connected to a fluid refrigerant supply unit 32 via one or more umbilicals 34a-c.

Referring now to FIG. 2, the cryotip (i.e. the distal portion) of the catheter 24 is shown in greater detail. As shown, the catheter 24 includes a catheter tube 36, contact segment 38 and a cryo-element 40. FIG. 2 also shows that the contact segment 38 extends from a distal end 42 (which is attached to the cryo-element) to a proximal end 44 (which is attached to the distal end 46 of the catheter tube 36). For the system 20, both the cryo-element 40 and the contact segment 38 are made of thermally conductive materials. Further, the cryo-element 40 is attached to the distal end 42 of the contact segment 38 to establish a thermally conductive interface therebetween which allows heat to flow between the contact segment 38 and cryo-element 40.

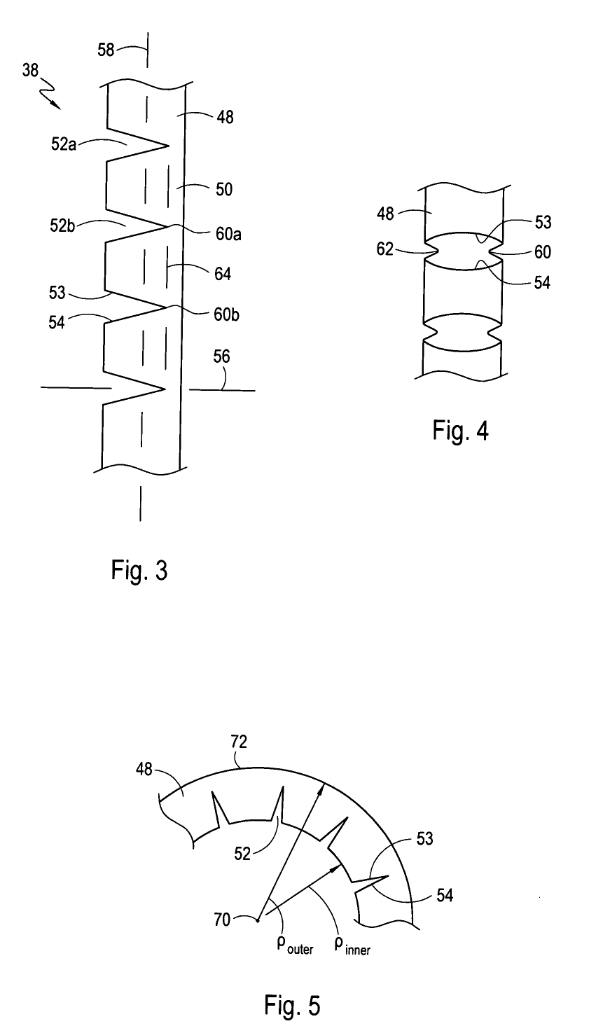

A better understanding of the contact segment 38 can be obtained with cross-reference to FIGS. 2 and 3. As seen there, the contact segment 38 includes an elongated, thermally conductive tube 48 that has an outer surface 50 and is formed with a plurality of transverse notches 52, for which exemplary notches 52a and 52b have been labeled. Typically, the tube 48 is made of a stainless steel material and the notches 52 are cut in the stainless steel tube 48 using a precision laser. As explained in more detail further below, the tube 48 is reconfigurable between a first configuration (shown in FIGS. 2 and 3) wherein the tube 48 is substantially cylindrical and a second configuration (shown in FIG. 5) in which at least a portion of the outer surface 50 of the tube 48 is shaped to substantially conform with the surface of the target tissue.

As best seen in FIGS. 3 and 4, each notch 52 establishes a first edge 53 and an opposed second edge 54. For the embodiment shown, each edge 53, 54 is inclined relative to a plane (such as a plane containing line 56) that is substantially perpendicular to a longitudinal axis 58 defined by the tube 48 when the tube 48 is in the first configuration (i.e. when the tube 48 is cylindrical shaped). It can further be seen that the first edge 53 and second edge 54 of each notch 52 meet at a respective first corner 60 and second corner 62.

For the embodiment shown, the notches 52 are arranged with their respective first corners 60a,b lying substantially along a common line, such as reference line 64, that extends parallel to the longitudinal axis 58 when the tube 48 is in the first configuration (as shown in FIG. 3). It is to be appreciated that for notches 52 of uniform shape and size, the second corners 62 will also lie along a common line that extends parallel to the longitudinal axis 58. This cooperation of structure allows the tube 48 to deflect in a single plane when reconfigured from the first configuration to the second configuration. However, those skilled in the pertinent art will recognize that by varying the shape, size and/or alignment of the notches 52, a tube 48 can be made to deflect in more than one plane. For example, the notches 52 can be arranged wherein a first section of the tube 48 deflects in a first plane and a second section of the tube 48 deflects in a second plane.

FIG. 5 shows the tube 48 after it has been reconfigured into the second configuration. As shown in FIG. 2, a pull-wire 66 attached to the distal end of the tube 48 at attachment point 68 can be actuated to reconfigure the tube 48 in the second configuration. When the tube 48 is configured as shown in FIG. 5, the first edge 53 of each notch 52 is juxtaposed with the second edge 54 of the notch 52, and the tube 48 is no longer cylindrical. For the exemplary embodiment shown in FIG. 5, the notches 52 are configured such that the tube 48 is curved in the second configuration and establishes an inner radius of curvature, ρᵢₙₙₑᵣ, relative to a central axis 70 and an outer radius of curvature, ρₒᵤₜₑᵣ, relative to the central axis 70. More specifically, the portion 72 of the tube 48 that is distanced from the central axis 70 by the distance ρₒᵤₜₑᵣ, constitutes a continuous, thermally conductive band that can be placed in contact with target tissue having a curved surface and cooled to cryoablate the target tissue.

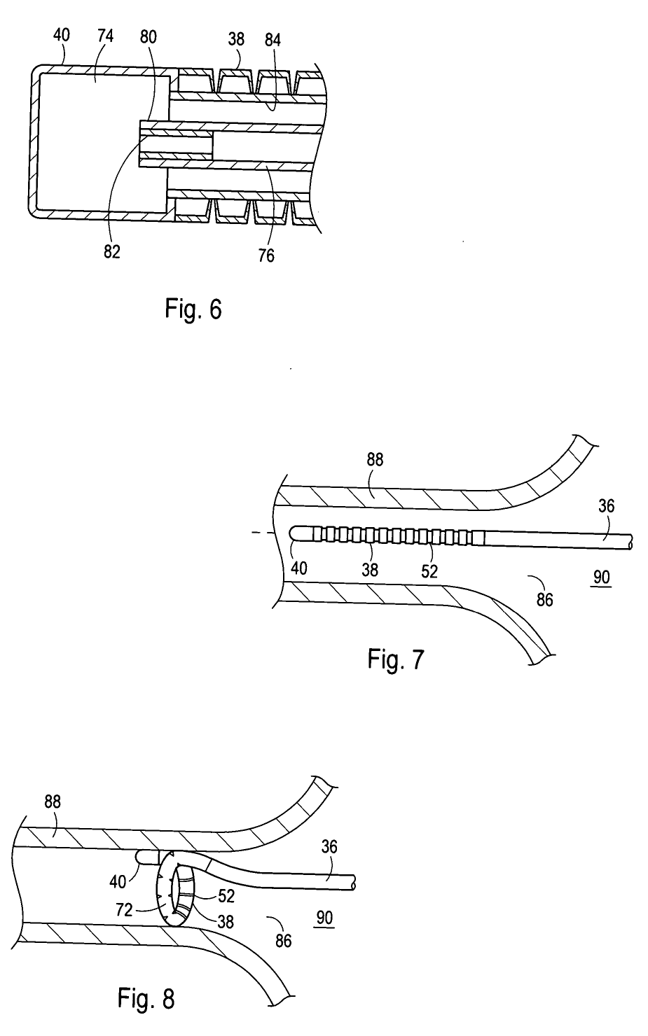

A more detailed understanding of the interactive cooperation between the contact segment 38 and the cryo-element 40 can be obtained with reference to FIG. 6. As shown, the cryo-element 40 surrounds and defines an expansion chamber 74. A supply tube 76 is provided that extends from a proximal end 78 to a distal end 80. As shown in FIG. 1, the proximal end 78 of the supply tube 76 is connected to a refrigerant supply unit 32 via umbilical 34a. Cross-referencing FIGS. 1 and 6, it can be seen that from the proximal end 78, the supply tube 76 passes through the handle 30, the catheter tube 36, the contact segment 38 and projects slightly into the expansion chamber 74. A restriction 82 can be positioned in the supply tube 76 at the distal end 80 to restrict the flow of refrigerant. A refrigerant return line 84 is arranged co-axially with the supply tube 76 to direct expanded refrigerant from the expansion chamber 74 to the refrigerant supply unit 32. Alternative arrangements (not shown) can include locating the cryo-element 40 at the proximal end 44 of the contact segment 38, or locating cryo-elements 40 at both the distal end 42 and the proximal end 44 of the contact segment 38.

### Operation

The operation of the system 20 can best be appreciated with reference to FIGS. 7 and 8 which show a treatment site at the ostium 86 of a pulmonary vein 88 where the pulmonary vein 88 connects to the left atrium 90. Referring to FIG. 7, the contact segment 38 is initially placed in the first configuration in which the contact segment 38 is cylindrical and somewhat straight. This configuration allows the distal portion of the catheter 24 to be somewhat easily navigated through the vasculature to the treatment site. During transit through the vasculature, a curve can be imparted to the contact segment 38 (using the pull-wire 66 shown in FIG. 2) to steer the distal portion of the catheter 24 to the treatment site. The catheter tube 12 is used to advance the contact segment 38 to the treatment site. At the treatment site, the distal portion of the catheter 24 is positioned near the target tissue to be cryoablated.

At the treatment site, the pull-wire (FIG. 2) can be activated to reconfigure the contact segment 38 in the second configuration, such as the configuration as shown in FIG. 8. In the second configuration, the portion 72 of the contact segment 38 is shaped as a continuous, thermally conductive band that can be placed in contact with a circumferential band of tissue surrounding the ostium 86 of a pulmonary vein 88 where the pulmonary vein 88 connects with the left atrium 90.

Once the contact segment 38 has been positioned at the treatment site, configured in the second configuration and placed in contact with the target tissue, a fluid refrigerant, such as Nitrous Oxide, from the refrigerant supply unit 32 is transferred through the supply tube 76 and into the expansion chamber 74 (FIG. 6) of the cryo-element 40. Inside the expansion chamber 74, the fluid undergoes endothermic expansion to absorb heat from the cryo-element 40 (and the contact segment 38 and target tissue). Typically, a fluid refrigerant is used that transitions from a liquid state to a gaseous state as it expands into the expansion chamber 74. Heat absorbed by the refrigerant during this phase transition (i.e. latent heat) cools the cryo-element 40, which in turn cools the contact segment 38, which cools and cryoablates the target tissue. After expansion, the gaseous fluid refrigerant can pass through the return line 84 (FIG. 6) and exit the patient 22 (FIG. 1).

After the target tissue has been cryoablated, the contact segment 38 can be warmed and reconfigured (using the pull-wire 66) to place the contact segment 38 into the first configuration (as shown in FIG. 7). For example, the contact segment 38 can passively absorb ambient heat at the treatment site to warm the contact segment 38. It will be appreciated, however, that the contact segment 38 can also be warmed by any other devices or methods known to those skilled in the pertinent art. Once in the first configuration, the contact segment 38 can then be withdrawn from the treatment site and removed from the patient.

While the particular articulating catheter tip with wedge-cuts as herein shown and disclosed in detail is fully capable of obtaining the objects and providing the advantages herein before stated, it is to be understood that it is merely illustrative of the presently preferred embodiments of the invention and that no limitations are intended to the details of construction or design herein shown other than as described in the appended claims.

1. An articulating catheter for cryoablating target tissue, the target tissue having a curved surface at a treatment site, said catheter comprising:
an elongated, thermally conductive tube having an outer surface and formed with a plurality of transverse notches, with each said notch establishing opposed first and second edges;
means for advancing said tube to the treatment site;
means for reconfiguring said tube from a first configuration wherein said tube is substantially cylindrical and defines a longitudinal axis and each edge is inclined relative to a plane that is substantially perpendicular to said axis, to a second configuration wherein each said first edge is juxtaposed with a respective said second edge to shape at least a portion of the outer surface of said tube to substantially conform with the surface of the target tissue; and
means for cooling said tube to cryoablate the target tissue.

2. An articulating catheter as recited in claim 1 wherein said first and second edges of each said notch meet at first and second corners and said notches are arranged with said first corner of each notch lying substantially along a common line that extends parallel to said longitudinal axis when said tube is in said first configuration.

3. An articulating catheter as recited in claim 1 wherein said tube is made of stainless steel.

4. An articulating catheter as recited in claim 1 wherein said advancing means comprises a catheter tube.

5. An articulating catheter as recited in claim 1 wherein said plurality of notches comprises at least three said notches.

6. An articulating catheter as recited in claim 1 wherein said reconfiguring means comprises a pull-wire.

7. An articulating catheter as recited in claim 6 wherein said tube extends from a proximal end to a distal end and said pull-wire is attached to said distal end of said tube.

8. An articulating catheter as recited in claim 1 wherein said cooling means comprises a cryo-element having an expansion chamber for expanding a refrigerant therein.

9. A reshapeable contact segment for use in a catheter for cryoablating target tissue having a curved surface, said contact segment comprising:
an elongated, thermally conductive tube having an outer surface and formed with a plurality of transverse notches, with each said notch establishing opposed first and second edges, wherein said tube is reshapeable between a first cylindrical configuration in which each edge is inclined relative to a plane that is substantially perpendicular to a longitudinal axis defined by the cylindrical tube, and a second configuration in which each said first edge is juxtaposed with a respective said second edge to shape at least a portion of the outer surface of said tube to substantially conform with the surface of the target tissue.

10. A reshapeable contact segment as recited in claim 9 wherein said first and second edges of each said notch meet at first and second corners and said notches are arranged with said first corner of each notch lying substantially along a common line that extends parallel to said longitudinal axis when said tube is in said first configuration.

11. A reshapeable contact segment as recited in claim 9 wherein said tube is made of stainless steel.

12. A reshapeable contact segment as recited in claim 9 wherein said plurality of notches comprises at least three said notches.

13. A reshapeable contact segment as recited in claim 9 wherein said segment further comprises a pull-wire having a distal end and a proximal end with said distal end attached to said tube.

14. A reshapeable contact segment as recited in claim 13 wherein said tube extends from a proximal end to a distal end and said pull-wire is attached to said distal end of said tube.

15. A method for cryoablating target tissue, the target tissue having a curved surface at a treatment site, said method comprising the steps of:
providing an elongated, thermally conductive tube having an outer surface and formed with a plurality of transverse notches, with each said notch establishing opposed first and second edges;
configuring said tube into a first configuration wherein said tube is substantially cylindrical and defines a longitudinal axis and each edge is inclined relative to a plane that is substantially perpendicular to said axis,
advancing said tube to the treatment site;
configuring said tube into a second configuration wherein each said first edge is juxtaposed with a respective said second edge to shape at least a portion of the outer surface of said tube to substantially conform with the surface of the target tissue;
contacting the target tissue with said tube; and
cooling said tube to cryoablate the target tissue.

16. A method as recited in claim 15 wherein said first and second edges of each said notch meet at first and second corners and said notches are arranged with said first corner of each notch lying substantially along a common line that extends parallel to said longitudinal axis when said tube is in said first configuration.

17. A method as recited in claim 15 wherein said tube is made of stainless steel.

18. A method as recited in claim 15 wherein said advancing step is accomplished using a catheter tube.

19. A method as recited in claim 15 wherein said plurality of notches comprises at least three said notches.

20. A method as recited in claim 15 wherein reconfiguring step is accomplished using a pull-wire.
